(19) United States
(12) Patent Application Publication
(10) Pub. No.: US 2013/0266627 A1
(43) Pub. Date: Oct. 10, 2013

(54) Method for Treating Mammals for Uterine Disorders with Therapeutic Nanolipidic Vehicles

(71) Applicants: John D. McCarroll, Pilot Point, TX (US); Kelly M. Miller, Clearwater, FL (US)

(72) Inventors: John D. McCarroll, Pilot Point, TX (US); Kelly M. Miller, Clearwater, FL (US)

(73) Assignee: DERMAZONE SOLUTIONS, INC., St. Petersburg, FL (US)

(21) Appl. No.: 13/857,362

(22) Filed: Apr. 5, 2013

Related U.S. Application Data
(60) Provisional application No. 61/793,668, filed on Mar. 15, 2013.
(60) Provisional application No. 61/622,105, filed on Apr. 10, 2012.

(51) Int. Cl. A61K 9/50; A61K 36/53

(52) U.S. Cl. CPC A61K 9/50; A61K 36/53
USPC 424/408; 424/725

(57) ABSTRACT

A composition for intrauterine lavage comprising phenolic compounds encapsulated within a potentiated nanolipidic process is disclosed. The encapsulated nanolipidic preparation is preferably provided as a concentrated solution. Said concentrated solution is mixed with a sterile diluent suitable for the intrauterine environment, and said diluted nanolipidic preparation is used to lavage the uterus of a mammal in need of treatment of one or more conditions of the uterus and/or to maintain general uterine health. Preferably, the encapsulated phenolic compounds are extracted from Origanum vulgare.

### CROSS REFERENCE TO RELATED APPLICATIONS

This application claims the benefit of U.S. Provisional Application Nos. 61/622,105, filed Apr. 10, 2012, and 61/793,668, filed Mar. 15, 2013.

### STATEMENT REGARDING FEDERALLY SPONSORED RESEARCH OR DEVELOPMENT

Not applicable.

### TECHNICAL FIELD OF INVENTION

This invention relates to the field of treatment of the uterus of mammals for disorders associated therewith.

### BACKGROUND OF THE INVENTION

The uterus of a mammal is subject to a variety of disorders. These disorders can cause further problems with respect to the health of the animal, as well as the reproductive capabilities of that animal.

The mammalian uterus may be subject to microbial infection, tissue inflammation, and irritation related to said microbial infection and tissue inflammation. Oftentimes, microbial infections are refractory to treatment with existing treatment protocols, such that the mammal's reproductive status remains compromised for an extended period of time. There remains a need for a treatment option which can alleviate said infection and inflammation, without causing further irritation of the uterine tissue.

Incorporating passenger molecules, such as pharmaceutical active ingredients, in lipid vesicles such as liposomes, has been reported in the prior art. An amphipathic carrier structure denoted as a Solvent Dilution Microcarrier (“SDMC”) was disclosed in U.S. Pat. No. 5,269,979. In general, the '979 patent described making a plurality of SDMC vehicles by solubilizing an amphipathic material and a passenger molecule in a first quantity of a non-aqueous solvent. Following this, a first quantity of water was added, forming a turbid suspension. In a subsequent step, a second quantity of non-aqueous solvent was added to form an optically clear solution. The final step of a preferred embodiment was to organize the optically clear solution into SDMC vehicles by mixing with air or a second quantity of water.

In U.S. Pat. No. 5,879,703, a method for preparing a shelf-stable precursor solution useful for remote encapsulation of active ingredients was described. In '703, the precursor solution was made by solubilizing an amphipathic material in a non-aqueous solvent. Although SDMCs and the shelf-stable precursor solution provided for making vehicles suitable for delivering active ingredients in a variety of applications, a need remained for improved vehicles for delivery of passenger molecules. The '979 Patent and the '703 Patent simply do not fill all the needs in the market or technology for encapsulated ingredients and components.

### SUMMARY OF THE INVENTION

A new treatment option comprises encapsulating an antimicrobial/anti-inflammatory active ingredient as a passenger molecule in a potentiated nanolipidic process capable of delivering said active ingredient to the uterine tissues of an animal in need of such treatment. A composition and method for intrauterine lavage is disclosed. Said composition has anti-bacterial, anti-fungal, and anti-viral activity, in addition to anti-inflammatory properties. Said method utilizes said composition for intrauterine lavage.

A composition comprising phenolic compounds encapsulated within a potentiated nanolipidic process is disclosed. The encapsulated nanolipidic preparation is provided as a concentrated solution. Said concentrated solution is mixed with a sterile diluent suitable for the intrauterine environment, and said diluted nanolipidic preparation is used to lavage the uterus of a mammal in need of treatment for one or more conditions of the uterus and/or to maintain general uterine health. Preferably, the phenolic compounds are extracted from Origanum vulgare.

A method of utilizing said encapsulated nanolipidic preparation in the treatment of uterine disorders in large mammals is also disclosed. The starting material for the encapsulated nanolipidic preparation is manipulated by dilution with a non-aqueous solvent, either before or after loading with a passenger molecule, to provide one or more defined populations of nanolipidic particles (“NLPs”) which range in size from about 1 nanometer to about 20 nanometers. NLP assemblies are formed from the NLPs which range in size from about 30 nanometers to about 200 nanometers. In addition, it has been found that NLPs can be used in a method for making carrier vehicle preparations which are mixed smaller and larger carrier vehicles, or having a larger mean size of about 200-300 nanometers, but improved encapsulation of passenger molecules. Disorders which may benefit from said method include, but are not limited to, post-partum metritis, endometritits and pyometria.

### DETAILED DESCRIPTION

Intrauterine lavage is performed in large animals during the post-partum stage to restore the uterine environment. Lavage may also be used to prepare the uterine environment prior to pregnancy. Intrauterine lavage may also be used to treat uterine disorders, such as infection, in animals.

An intrauterine lavage composition, and method for administration of said composition to mammals, is herein described, which provide beneficial results to animals in need of such treatment. The invention is particularly beneficial for use in horses, cattle, sheep and other mammalian livestock.

Uterine lavage is a procedure used to flush, irrigate or otherwise wash out the interior of the uterus using a volume of fluid. Uterine lavage is beneficial in the treatment and prevention of uterine disorders in animals. A lavage may be used to irrigate, or wash the interior of the uterus to provide a cleansing effect, or a lavage may be used to administer a therapeutic agent to the interior of the uterus; a lavage can also provide the combined effect of cleansing the uterus while delivering a therapeutic agent to the uterine interior.

A lavage aids in the treatment of uterine infections by removing exudate or retained materials from the uterus, and may also stimulate uterine contractions which aid in clearing the contents from the uterus. Lavage may also be used as a preventative treatment against contamination in the uterus or may be used to prepare the uterine environment prior to breeding.

A lavage for irrigating the uterus to remove exudate or retained materials may be performed using a large volume of fluid such that all surfaces of the uterine interior are contacted with the lavage fluid. Fluid used for large volume lavage may also contain therapeutic compounds useful in the treatment of uterine disorders. The fluid volume may then be removed by catheter, gravity flow, uterine contraction, or by other means suitable for evacuating the uterine interior.

Low volume lavage may also be used to introduce therapeutic compounds to the uterine environment. Low volumes of lavage preparations may be introduced to the uterine environment in prescribed dosing amounts to treat uterine disorders, to treat the uterus prophylactically for contamination, or to prepare the uterus prior to breeding. The fluid volume may then be removed by catheter, gravity flow, uterine contraction, or by other means suitable for evacuating the uterine interior.

Aseptic techniques should be used in the preparation and administration of the lavage composition. The lavage composition of the invention may either be provided in a concentrated form requiring dilution of the concentrate with a sterile diluent prior to use, or may be provided as a ready-to-use preparation which does not required dilution prior to use.

Example steps for using the concentrated lavage composition include: 1) aseptic dilution of the lavage composition with a sterile diluent, such as sterile water, saline or Lactated Ringers solution; 2) administering diluted lavage composition to the uterine environment utilizing an apparatus, such as, but not limited to, a catheter; 3) evacuating the lavage composition from the uterus. Evacuation of the lavage composition may be by catheter, gravity flow, or by uterine contractions, depending upon circumstances specific to the animal.

A ready-to-use preparation of the lavage composition, which preparation does not required dilution prior to administration, may also be used by: 1) administering the lavage composition to the uterine environment utilizing an apparatus, such as, but not limited to, a catheter; and 2) evacuating the lavage composition from the uterus. Evacuation of the lavage composition may be by catheter, gravity flow, or by uterine contractions, depending upon circumstances specific to the animal.

The lavage composition of the invention may be administered in a dose-wise manner, wherein a specific volume is administered, such as 100 mL of diluted preparation per dose, and dosing may be repeated as needed.

Alternatively, the lavage composition may be used in a larger volume, such that the lavage composition is administered until all surfaces of the uterine interior are sufficiently contacted with the lavage composition to provide effective irrigation or washing of the uterine interior, and the fluid starts to evacuate from the uterus. The volume of lavage composition to administer until the fluid starts to evacuate would be largely dictated by the size of the animal being treated and size of the animal's uterus.

The lavage composition of the invention comprises antimicrobial organic phenolic compounds encapsulated in nanolipidic vehicles. The organic phenolic compounds are preferably obtained from plant oil extracts from the Labiatae family. The lavage composition preferably also comprises non-phenolic botanicals extracted from said plant oil. In an alternative embodiment, synthetically derived forms of said antimicrobial organic phenolic compounds may be used to provide or augment antimicrobial properties.

The antimicrobial organic phenolic compounds obtained from Origanum vulgare are preferably combined with lecithin, ethanol and water to form nanolipidic particles with said antimicrobial phenolic compounds encapsulated within. Most preferably, soy lecithin is employed. The encapsulated organic phenolic compounds may be used in a composition for uterine lavage which exhibits little or no irritation, discomfort or unpleasant side-effects to the treated animal.

Plant oil extracts from the Labiatae family, preferably Origanum vulgare, are obtained as commercially-available natural oil.

The Origanum vulgare extract has antimicrobial properties, including anti-viral, anti-bacterial, and anti-fungal activity. In addition, the extract also has anti-inflammatory properties which are beneficial to the uterine environment since inflammation generally accompanies microbial infection.

The extract from Origanum vulgare comprises carvacrol and thymol, two phenolic compounds. A suitable extract for use in preparing the uterine lavage composition of the invention preferably has a concentration of phenolic compounds in the extract of 50% to 75%. Within the total, the concentration of thymol is from about 3.0 to 9.5% and the concentration of carvacrol is within a range 47.0 to 65.5%.

Standardized concentrations of thymol and carvacrol are used for preparation of the nanolipidic vehicles used to prepare the composition of the invention. Gas chromatography (GC) or High Performance Liquid Chromatography (HPLC) may be used to verify that the concentration of thymol and carvacrol in the extract is within the therapeutically effective concentration range.

Nanolipidic particles (“NLP”s) according to the present invention have a size range of from about 1 to about 20 nanometers, as measured by a standard laser light scattering technique, discussed in detail herein. Various subpopulations of NLPs may be made. The preferred distinct subpopulations of NLPs range in size from about 1 to about 4 nm, from about 4 to about 7 nm, from about 7 to about 10 nm, from about 10 to about 14 nm, from about 14 to about 18 nm, and from about 18 to about 20 nm. A preferred subpopulation comprises NLPs having an average size of about 9 to 10 nanometers.

### Preparation of Nanolipidic Particles (NLPs) and NLP Assembly Populations

Preloaded nanolipidic particles (NLPs) are prepared according to the techniques set forth in United States Patent Application Publication. No. 2010/0239686 A1, published Sep. 23, 2010, and United States Patent Application Publication No. 2012/00195940 A1, published Aug. 2, 2012, which are herein incorporated by reference. NLPs are prepared from a Shelf-Stable Precursor Stock, prepared according to U.S. Pat. No. 5,879,703 which is herein incorporated by reference.

NLPs are made from a precursor solution as described in U.S. Pat. No. 5,879,703, which is herein incorporated by reference as if fully set forth herein. As stated in the '703 patent, a precursor solution may be made by solubilizing an amphipathic material in a first quantity of a non-aqueous solvent appropriate to solubilize the amphipathic material to form a first mixture. The amphipathic material preferably comprises phospholipids (PL). A preferred PL comprises one or more of the following phosphatides: phospatidylcholine (PC), phospatidylethanolamine (PE), phosphatidic acid (PA) and phosphatidylinositol (PI). In a preferred embodiment, PC, PE, PA and PI are combined. A preferred ratio of phospholipids useful in the invention is PC:PE:PA:PI of 6.5:2.5:0.7:0.3 in ethanol. Preferably, one gram of PL is solubilized in 5.0-7.5 mL of ethanol solvent.

After dissolution of the amphipathic material, a quantity of water is added to form a turbid suspension. The amount of water to add is approximately 9 kg water to 31 kg of dissolved amphipathic material, but can be varied to result in the desired turbid suspension. A second quantity of non-aqueous solvent, such as ethanol, is added until the turbid suspension is monophasic and has optical clarity at room temperature. This resulting product is a precursor solution which is shelf-stable over time.

In the '703 patent, it was disclosed that a precursor solution made according to the process disclosed therein was shelf stable at least up to two years, and perhaps longer, as long as it remains in a monophasic condition. It has been recently determined that precursor solutions made by this method are stable for at least eight years, independent of manufacturing, location, season, year and lot.

It has now been found that a precursor solution such as disclosed in '703 can be used as a starting material to make nanolipidic particles (NLPs) and NLP assemblies. In '703, the precursor solution was disclosed as being useful for making SDMCs at a later point in time and, perhaps, a remote location. SDMCs have a diameter of from about 230 to about 412 nm. In contrast, NLPs have a mean diameter of from about 1 nm to about 20 nm and NLP assemblies have a mean diameter from about 30 nm to about 200 nm.

Various populations of NLP assemblies may be made for various applications. Preferred populations range from about 40-60 nm; about 60-80 nm; about 80-110 nm; about 110-140 nm; and about 150-200 nm. NLP assembly populations are generally 20-30% smaller in diameter than SDMCs for the same passenger molecule.

A slightly larger population or mixed population of carrier vehicles is referred to herein as ECVs or encapsulating carrier vehicles. Although overlapping the mean diameter of SDMCs, the ECV is made using a different method employing NLPs and the result is a carrier vehicle population which has been found to exhibit a higher encapsulating efficiency. The ECVs are described as having a mean diameter from about 200 nm to 300 nm.

To make carriers for passenger molecules, such as NLP populations, NLP assemblies, or ECVs according to the methods disclosed in United States Patent Application Publications No. 2010/0239686 and 2012/0195940, such as NLP populations, NLP assemblies, or ECVs, the precursor solution as previously described in the '703 patent is diluted with a suitable solvent or mixed solvent system which is compatible with the solvent system used in the precursor solution.

This dilution is performed either before or after addition of the passenger molecule as will be further described in detail below. The solvent is selected for biocompatibility if the end use of the carriers will require that characteristic. The solvent or mixed solvent system used for dilution must be miscible with the solvents in the precursor solution and should be effective to disperse rather than dissolve the carriers. Most preferably, the solvent used for dilution is ethanol, since it possesses the desired qualities. The dilution is preferably conducted in a sequential or serial manner. For example, a first dilution of 1:10 provides a population of carriers, and further serial dilution to about 1:0.5 provides a series of populations of carriers.

The size of the carriers in each dilution can be determined by laser light scattering. Mixed populations of NLPs and larger vesicles may be created at lower dilutions with the non-aqueous solvent. An appropriate instrument for this purpose is the Zetasizer 1000 manufactured by Malvern Instruments, (Worcestershire United Kingdom). Diameters of particles reported herein were determined using the Multimodal Analysis Mode of the Zetasizer 1000 to determine particle size by peak intensities. Other techniques may be used to analyze particle size, which results can be correlated to the numerical values obtained with the light scattering technique described herein.

Addition of the desired passenger molecule occurs prior to dilution with the solvent if the passenger molecule is lipophilic or amphipathic. Addition occurs after dilution if the passenger molecule is water soluble.

Thus, in the case of a lipophilic or amphipathic passenger molecule, the NLP loaded populations form upon dilution with the solvent. NLP assembly populations or ECVs are formed by dilution of the NLP loaded population into water.

In the case of a water soluble passenger molecule, the precursor solution is mixed with a passenger molecule dissolved in water. NLP assembly populations or ECVs are formed upon dilution with the non-aqueous solvent. If a serial dilution technique is used, distinct populations are formed.

Based on curves observed from different classes of compounds, ranges for the finished NLP assembly population can be established for each NLP population used to form the final NLP assembly population. The more non-aqueous solvent that is used to dilute the NLPs, the smaller the NLP assembly populations.

Various NLP loaded populations may be mixed and matched to provide a multifunctional NLP assembly product. The different NLP loaded populations within the NLP assembly could provide a preparation which allows one active ingredient to be preferentially absorbed over the other, thus allowing a control of the rates of penetration of different ingredients in a single preparation. Alternatively, a single NLP population could be loaded with more than one passenger molecule to provide the multifunctionality. For example, the NLP preparations disclosed herein have phenolic compounds encapsulated within the vesicles for treatment of the uterine environment, but the preparations could alternatively be loaded with a secondary passenger molecule to provide additional benefit to the uterine environment.

The ability to select an NLP population of a preferable size for a given application provides advantages to the manufacturing process as well. Less material will be required to form the end product if an NLP precursor solution is selected for a particular size need. Loading efficiency also goes up. The number of NLP particles increases as the size of the NLP populations decreases as a function of the decreasing diameter of the spherical NLP product (assuming the amount of lipid to passenger molecule remains constant). This may provide a higher concentration of passenger molecule per unit volume.

The passenger molecules suitable for use in forming a NLP loaded population are numerous. In one embodiment, passenger molecules can be selected which exhibit lipid solubility or are amphipathic. These molecules have solubility profiles ideally suited for loading into NLPs. In another embodiment, water soluble molecules may be incorporated into NLPs by solubilization into the aqueous solution used to form the finished NLP product. Using these two approaches virtually any molecule may be incorporated as a passenger molecule into NLP products of defined sizes. An innovative use of both approaches may be used to incorporate both lipid and water soluble compounds into a NLP assembly product by first incorporating lipid soluble compounds into NLPs prior to dilution with ethanol and second incorporating water soluble molecule(s) into the water solution used to form the finished NLP product of defined size.

Numerous passenger molecules have been incorporated into NLPs. For example, fat-soluble vitamins may be used as passenger molecules. Vitamins D, E and K have been found to be appropriate for NLPs.

Water soluble vitamins, such as Vitamin B and C may also be used as passenger molecules. Both water soluble and fat soluble vitamins may be combined in an NLP assembly if it is desired that both be administered by using the technique discussed above.

Other preferred passenger molecules are antibiotics such as aminoglycosides (Gentamycin), beta-lactams (Penicillin G) and macrolides (Erythromycin). Anesthetics such as lidocaine have been effectively incorporated in NLPs, as have steroids and antifungals (griseofulvin).

NLPs may also be used for incorporation of peptides. Tripeptides, Tetrapeptides, Hexapeptides and Nonapeptides have been effectively incorporated into NLPs.

If the desired passenger molecule is water soluble, the passenger molecule should first be dissolved in water. The incorporation step, or loading of the passenger molecule into the NLP, is accomplished when the NLP product is formed by adding the dissolved passenger molecule to the precursor solution.

### Example Preparation of NLPs and NLP Assembly Populations with Encapsulated Phenolic Compounds

NLPs containing lipid-soluble phenolic compounds were prepared as follows:

Solvent diluted precursor stock was prepared by adding 1 part shelf-stable precursor stock to 0.3 part ethanol.

An aliquot of extract of Origanum vulgare comprising 3.0 to 9.5% of thymol and 47.0 to 65.5% of carvacrol was dissolved in the solvent diluted precursor stock. This solution was stirred at room temperature resulting in a preloaded NLP population

The preloaded NLP preparation was diluted into distilled water to yield a liposomal concentrate comprising thymol in the amount about 0.01% to 0.07% and carvacrol in the amount of about 0.1% to 0.5%.

The liposomal concentrate for the lavage composition of the invention preferably comprises thymol in the amount of about 0.001% to 0.10% and carvacrol in the amount of 0.01% to about 1.0%. Most preferably, the concentrate comprises thymol in the amount of about 0.01% to 0.07% and carvacrol in the amount of about 0.1% to 0.5%.

The size of the preloaded NLPs may be determined by using the Malvern 1000 Zetasizer Laser Light Scattering Instrument set to analyze populations using multimodal analysis mode. The size of the finished preparation was determined to be 140-170 nm.

Nanolipid particle sizes for the composition of the invention can be increased or decreased by adjusting the ratio of ethanol to Solvent Dilution Microcarrier (SDMC) (U.S. Pat. No. 5,879,703) used in preparation of the precursor stock solution. Particle sizes can range from approximately 60 nm using 20 parts ethanol: 1 part SDMC and up to 170 nm using 0.3 part ethanol: 1 part SDMC.

One or more additional dilutions of the precursor solution may be made with ethanol solvent in order to provide a desired size of NLPs and number of NLPs per unit volume. If a serial dilution technique is used, distinct populations are formed. The more non-aqueous solvent that is used to dilute the NLPs, the smaller the NLP assembly populations.

Nanolipidic particles sizes useful for the lavage composition of the invention are from about 20 nm to 300 nm, preferably from 30 nm to 200 nm and most preferably from 60 nm to 170 nm.

### Diluted Nanolipid-Encapsulated Phenolic Concentrate for Use as Intrauterine Lavage

A composition suitable for intrauterine lavage is prepared for administration by aseptically diluting the concentrated nanolipid-encapsulated antimicrobial phenolic preparation with suitable sterile diluents, such as water, Lactated Ringer's solution (consisting of 130 mmol/L sodium ion, 109 mmol/L chloride ion, 28 mmol/L lactate, 4 mmol/L potassium ion and 1.5 mmol/L calcium ion), and saline solution.

The composition is useful as an intrauterine lavage for animals, including, but not limited to, cattle, horses and sheep.

The composition for intrauterine lavage is particularly efficacious for use in beef and dairy cattle, but is not limited to cattle. Horses, sheep, goats, as well as other animals, may also receive intrauterine lavage with the composition.

The composition allows for optimal absorption of therapeutic agents into the uterine lining to restore or maintain the uterine environment. The composition has superior penetrating properties because the antimicrobial phenolic compounds are encapsulated in a potentiated liposomal process, allowing the active phenolic ingredients to affect not only the surface contacted tissues, but also to penetrate to subcutaneous and submucosal tissues as well. The composition is non-irritating, long-lasting and readily absorbed into the uterine lining leaving no drug residue. No milk withholding period is required following administration of the preparation.

The composition is antimicrobial and is efficacious in the eradication of microbes which may be infecting the uterus. The composition is particularly advantageous when microbial infections have become resistant to conventional antibiotics, or when secondary opportunistic infective agents are present in the uterus.

By providing a composition that can be administered to the uterus through lavage, the antimicrobial compounds are put in direct contact with the microbes. The nanolipidic vehicles are not irritating to sensitive uterine tissue, yet provide a timed-release of the antimicrobial phenolic compounds. The lavage composition can be used in a dose-wise manner using a low volume of fluid to place the antimicrobial phenolic compounds in contact with the uterine tissue. The lavage composition can also be used as a large volume lavage for washing or irrigating the interior of the uterus while allowing the antimicrobial phenolic compounds to contact all of the interior surfaces of the uterus.

In one embodiment, the liposomal preparation of the invention may be used as a post-partum lavage for animals in the following manner: 50 mL of the concentrated liposomal preparation is diluted in 5000 mL of sterile diluent, such as saline, Lactated Ringers or water. Intrauterine lavage is performed post-partum using 100 mL of diluted preparation per animal. Lavage may be repeated with additional 100 mL doses administered, as needed, to restore uterine health. Alternatively, the lavage may be administered in a larger volume such that all of the interior surfaces of the uterus are contacted with the lavage composition.

In another embodiment, the liposomal preparation of the invention may be used as a pre-pregnancy lavage for animals to optimize fertility by treating endometritis in the following manner: 50 mL of the concentrated liposomal preparation is diluted in 5000 mL of sterile diluent, such as saline, lactated ringers or water. Lavage can be administered dose-wise, as needed, to restore uterine health, with a preferred dose being in 100 mL. Alternatively, the lavage may be administered in a larger volume such that all of the interior surfaces of the uterus are contacted with the lavage composition. Lavage may be repeated as necessary.

In another embodiment, the liposomal preparation of the invention may be used to optimize fertility in mammals as a pre-breeding conditioning lavage in the following manner: 50 mL of the concentrated liposomal preparation is diluted in 5000 mL of sterile diluent, such as saline, lactated ringers or water. Lavage can be administered dose-wise, as needed, to restore uterine health, with a preferred volume of 100 mL. Alternatively, the lavage may be administered in a larger volume such that all of the interior surfaces of the uterus are contacted with the lavage composition. Lavage may be repeated as necessary.

In another embodiment, the liposomal preparation of the invention may be used in animals immediately post-partum as a prophylactic lavage for prevention of uterine contamination or infection. 50 mL of the concentrated liposomal preparation is diluted in 5000 mL of sterile diluent, such as saline, lactated ringers or water, and the diluted preparation may be either administered dose-wise as a low volume lavage or may be administered in a larger volume sufficient for contacting all of the interior surfaces of the uterus. Lavage may be repeated as necessary.

In yet another embodiment, the liposomal preparation of the invention may be used as a treatment for disorders of the uterus in post-partum animals, including, but not limited to pyometra and metritis. 10 mL of liposomal preparation is diluted in 100 mL sterile diluent, such as saline, Lactated Ringer's solution or water. The diluted preparation is then used as a uterine infusion; doses may be repeated until the animal is asymptomatic.

Alternatively, the liposomal composition of the invention is supplied in a ready-to-use sterile preparation, wherein dilution is not required prior to use. The ready-to-use preparation may be used in the any of the embodiments described herein, either dose-wise as a low volume lavage or administered in a larger volume sufficient for contacting all of the interior surfaces of the uterus.

### EXAMPLE 1

A mare, with pyometra that had been refractory to multiple therapy protocols, was treated with intrauterine lavage utilizing the intrauterine lavage composition of the invention. The mare had an immediate response to the therapy and was returned to functional reproductive status.

### EXAMPLE 2

Dairy cattle exhibiting post-partum metritis were infused with the intrauterine lavage composition of the invention. A significant improvement in conception rates resulted in the treated cattle.

We claim:

1. A composition for intrauterine lavage, comprising nanolipidic particles in which an extract of Origanum vulgare is encapsulated, said extract comprising antimicrobial phenolic compounds.

2. The composition of claim 1, wherein said antimicrobial phenolic compounds comprise thymol and carvacrol.

3. The composition of claim 1, wherein the concentration of said antimicrobial phenolic compounds in said extract is from about 50%-75% by volume of said extract.

4. The composition of claim 2, wherein the concentration of said thymol is from about 3.0% to 9.5% and the concentration of said carvacrol is from about 47.0% to 65.5% by volume of said extract.

5. The composition of claim 1, wherein said extract further comprises non-phenolic botanicals extracted from said Origanum vulgare.

6. The composition of claim 1, wherein said composition is a concentrated composition which is diluted prior to use as a lavage.

7. The composition of claim 6, wherein said concentrated composition has said antimicrobial phenolic compounds encapsulated at a concentration of about 0.011% to about 1.1%

8. The composition of claim 7, wherein said concentrated composition has said antimicrobial phenolic compounds encapsulated at a concentration of about 0.11%-0.57% by volume.

9. The composition of claim 6, wherein said concentrated composition is diluted with an aqueous diluent prior to use.

10. The composition of claim 9, wherein said aqueous diluent is a sterile aqueous solution suitable for lavage.

11. The composition of claim 9, wherein said aqueous diluent is selected from sterile water, sterile saline, and sterile Lactated Ringers solution.

12. The composition of claim 6, wherein 50 mL of said concentrated composition is diluted in 5000 mL of sterile diluent.

13. The composition of claim 6, wherein 10 mL of said concentrated composition is diluted in 100 mL of sterile diluent.

14. The composition of claim 6, wherein 5 mL of said concentrated composition is diluted in 100 mL of sterile diluent.

15. The composition of claim 1, wherein said composition is in a ready-to-use preparation such that said composition does not require dilution prior to use as a lavage.

16. A method of preparing a composition comprising nanolipidic particles with encapsulated antimicrobial phenolic compounds for use as an intrauterine lavage, comprising the steps of:
(a) providing a monophasic precursor solution;
(b) adding an extract of Origanum vulgare comprising antimicrobial phenolic compounds to said monophasic precursor solution to form a loaded nanolipidic particle population, wherein said loaded nanolipidic particles have said antimicrobial phenolic compounds encapsulated within said particles;
(c) diluting said loaded nanolipidic particle population with ethanol to form loaded nanolipidic particles, wherein said diluting of said loaded nanolipidic particle population with said ethanol solvent is at a predetermined ratio of loaded nanolipidic particle population to solvent, and
(d) adding an aliquot of said loaded nanolipidic particle population to an aqueous solvent to form a nanolipidic particle assembly population.

17. The method of claim 16, wherein said diluting of said precursor solution with said ethanol solvent is at a ratio of about 1 part precursor to about 20 parts solvent to a ratio of about 1 part precursor to about 0.3 parts solvent.

18. The method of claim 16, wherein the said nanolipidic particle assembly population has a mean particle diameter from about 20 nm to 300 nm.

19. The method of claim 18, wherein the said nanolipidic particle assembly population has a mean particle diameter from about 60 nm to 170 nm.

20. The method of claim 16, wherein said extract comprises antimicrobial phenolic compounds having a concentration of 50% to about 75% by volume.

21. The method of claim 16, wherein said antimicrobial phenolic compounds comprise thymol and carvacrol.

22. The method of claim 21, wherein the concentration of said thymol is from about 3.0% to 9.5% and the concentration of said carvacrol is from about 47.0% to 65.5% by volume of said extract.

23. The method of claim 16, wherein said precursor solution comprises phospholipids selected from the group consisting of phosphatidylcholine (PC), phosphatidylethanolamine (PE), phosphatidic acid (PA) and phosphatidylinositol (PI) and mixtures thereof.

24. The method of claim 16, wherein said phospholipids consist of a mixture of phosphatidylethanolamine (PE), phosphatidic acid (PA) and phosphatidylinositol (PI), wherein the ratio of said phospholipids in said mixture is PC:PE:PA:PI of 6.5:2.5:0.7:0.3.

25. The method of claim 16, comprising making one or more additional dilutions of said precursor solution with said ethanol solvent wherein said additional dilutions form distinct populations of nanolipidic particles, and wherein said nanolipidic particle populations decrease in size as ethanol concentration in precursor solution increases.

26. The method of claim 16, comprising making one or more additional dilutions of said precursor solution with said ethanol solvent in order to provide a desired number of nanolipidic particles per unit volume.

27. The method of claim 16, wherein said aqueous solvent further comprises a water-soluble passenger molecule.

28. The method of claim 16, wherein one or more additional lipophilic or amphipathic passenger molecules are added to said precursor solution to form a loaded nanolipidic particle population wherein said particles encapsulate admixed passenger molecules.

29. The method of claim 1, wherein said composition is a concentrated composition which is diluted prior to use as a lavage.

30. A method of intrauterine lavage, comprising the steps of:
a) providing a concentrated composition comprising nanolipidic particles with encapsulated extract of Origanum vulgare, said extract containing antimicrobial phenolic compounds;
b) aseptically diluting said concentrated composition with a sterile diluent suitable for use in a lavage; and
c) using diluted composition to lavage the uterus of a mammal in need of such treatment.

31. The method of claim 30, wherein said antimicrobial phenolic compounds comprise thymol and carvacrol.

32. The method of claim 30, wherein said concentrated composition has said phenolic compounds encapsulated at a concentration of about 0.011%-1.0% by volume.

33. The method of claim 32, wherein said concentrated composition has said phenolic compounds encapsulated at a concentration of about 0.11%-0.57% by volume.

34. The method of claim 30, wherein said concentrated composition is diluted with an aqueous diluent prior to use as a lavage.

35. The method of claim 34, wherein said aqueous diluent is a sterile aqueous solution suitable for lavage.

36. The method of claim 34, wherein said aqueous diluent is selected from sterile water, sterile saline, and sterile Lactated Ringers solution.

37. The method of claim 30, wherein 50 mL of said concentrated composition is diluted in 5000 mL of sterile diluent.

38. The method of claim 30, wherein 10 mL of said concentrated composition is diluted in 100 mL of sterile diluent.

39. The method of claim 30, wherein 5 mL of said concentrated composition is diluted in 100 mL of sterile diluent.

40. The method of claim 30, wherein said mammal is a ruminant.

41. The method of claim 30, wherein said mammal is a non-ruminant.

42. The method of claim 30, wherein said mammal is treated intrauterine for signs of infection.

43. The method of claim 30, wherein said mammal is treated after giving birth or expelling one or more fetuses.

44. The method of claim 30, wherein said mammal is treated for inflammation of the uterus.

45. The method of claim 30, wherein said mammal is treated to prepare the uterus for pregnancy.

46. The method of claim 30, wherein said mammal is treated prophylactically immediately post-partum.
